(19) United States
(12) Patent Application Publication
(10) Pub. No.: US 2004/0136953 A1
(43) Pub. Date: Jul. 15, 2004

(54) Methods of treating respiratory conditions

(76) Inventors: Paul Thomas King, Clifton Hill (AU); Stephen Roger Holdsworth, South Melbourne (AU)

Correspondence Address: ROTHWELL, FIGG, ERNST & MANBECK, P.C., 1425 K STREET, N.W., SUITE 800, WASHINGTON, DC, 20005, US

(21) Appl. No.: 10/471,405

(22) PCT Filed: Mar. 19, 2002
(86) PCT No.: PCT/AU02/00313

(30) Foreign Application Priority Data
Mar. 19, 2001 (AU) PR 3816

(51) Int. Cl. A61K 38/21

(52) U.S. Cl. 424/85.2; 424/85.6

(57) ABSTRACT

The present invention provides a method of treating a respiratory condition wherein said condition results from an infection by a pathogan and wherein said infection is characterised by a differing T helper cell response, said method including administering an effective amount of an agent to induce an equivalent T helper cell response which favours treatment of the respiratory condition.

The present invention relates to methods for treating respiratory conditions, in particular those conditions which may be characterised by a T helper cell response. Methods are also provided to identify such conditions. The invention further includes compositions useful for the treatment of the conditions.

### BACKGROUND

T helper (Th) cells direct the adaptive immune response. Th cells comprise 2 major subsets characterised by their cytokine profile and the pattern of immune effectors. Th1 cells produce IFN-γ, IL-2, IL-12 or IL-18, directing cell mediated immunity and are important in host defense against intracellular organisms, such as Tuberculosis and Legionella. Th2 cells produce IL-4, IL-5 and IL-10, directing humoral immunity and are important in host defense against extracellular organisms, such as Streptococcus.

The adaptive immune response is mediated by antigen specific Helper T (CD4⁺/T_{H}) cells. The concept of T_{H}1/T_{H}2 differentiation has been considered in coordinating adaptive immunity. T_{H}1 (cell mediated) responses are directed against intracellular pathogens while T_{H}2 (humoral) responses are directed against extracellular pathogens. Initiation of an inappropriate response can lead to unhindered spread of infection resulting in severe host pathology.

Differing Th1/Th2 responses have been described in several infectious diseases including Leishmaniasis, Schistosomiasis and Leprosy. In leprosy a Th1 response is associated with mild controlled disease against this intracellular organism, while a Th2 response (where antibody is produced that is ineffective against this intracellular organism) is associated with aggressive fatal disease. It has been postulated for these diseases that cytokine therapy with IFN-γ may cause a switch from a Th2 to a Th1 response with substantial clinical improvement. But this type of therapy is only a consideration when a differing Th1/Th2 response is identified and detected.

However, the nature of the Th cytokine response is not well established, either in normal or in those patients with chronic conditions. It is not evident which conditions react by switching a Th1 response to a Th2 response.

Present methods for measuring cytokine responses especially the Th1 or Th2 responses are laborious and time consuming with many techniques taking years to complete. The process involves growing and cloning cells which often change in behaviour after several passages in culture. This is not only undesirable for patients who have chronic infection and who wish to treat their conditions immediately but the result from highly passaged cells may not reflect the true in situ conditions to predict an appropriate treatment for the infection.

Respiratory conditions may be caused by any number of infections of which Nontypable Haemophilus influenzae (NTHi), is probably the most frequently encountered bacteria in respiratory practice. However its role has not been well defined. It has been found that:

i) NTHi is a universal bacteria to which most people have been exposed and is found in the nasopharnyx of up to 75% of healthy people.

ii) NTHi is a major cause of adult respiratory infection (as opposed to the capsulated types such as Hi type b). Recent work has shown that it invades the lung extensively in a variety of chronic lung diseases including COPD (chronic obstructive pulmonary disease), cystic fibrosis & bronchiectasis and lives intracellularly, especially in macrophages. It may have a particularly important role in the early pathogenesis of these conditions before the arrival of resistant bacteria, such as Pseudomonas. In addition it is also the major cause of childhood chronic tonsillitis.

iii) The nature of the Th cytokine response particularly with NTHi infection, has not been well established, either in normal subjects or in those with chronic lung disease.

iv) NTHi is involved in infections which lead to emphysema, ear infections, asthma, tonsillitis and bronchiectasis.

NTHi is an intracellular pathogen, and it may be expected that the appropriate protective response would be a Th1 response. However, chronic NTHi infections persist thereby suggesting an ineffective Th1 response. Patients with these infections appear to show normal immune systems. Many chronic infections persist for unexplained reasons, particularly when the immune system appears to be normal. However, it has not been apparent in these cases to consider the cytokine response, least of all distinguish between Th1 and Th2 responses.

Hence it is an object of the present invention to overcome or at least alleviate some of the problems of the prior art. In particular, it is an object to treat or arrest further infection in the respiratory tract.

### SUMMARY

In a first aspect of the present invention there is provided a method of treating a respiratory condition wherein said condition results from an infection by a pathogen and wherein said infection is characterised by a differing T helper cell response, said method including administering an effective amount of an agent to induce an equivalent T helper cell response which favours treatment of the respiratory condition.

The condition to be treated is preferably a respiratory condition, most preferably it is a respiratory condition of the upper and lower respiratory tract. This includes infections of the ear, nose and throat as well as the lungs.

Preferably the respiratory condition is selected from the group including chronic lung disease such as COPD, cystic fibrosis, bronchiectasis, tonsillitis, emphysema, ear infections or asthma. All of these conditions in their chronic form can be identified by standard symptoms including shortness of breath, coughing and severe bronchitis.

Most preferably, the pathogen is NTHi which causes any one of the conditions selected from the group including COPD, cystic fibrosis, bronchiectasis, emphysema, ear infection, asthma or tonsillitis. Even more preferred, the infection is NTHi causing bronchiectasis.

In the present invention, the method is applicable to a condition which is associated with an infection that is characterised by a differing T helper cell response. Preferably the differing T helper cell response is a difference between a Th-1 and a Th-2 response. These responses are characterised by their cytokine profiles and patterns of immune effectors. Generally the difference between the Th-1 and the Th-2 response is evident between a mild infection and an aggressive infection.

In a preferred aspect, there is provided a method of treating a NTHi infection, said method comprising administering an effective amount of an agent which induces an equivalent T helper cell response which favours treatment of the NTHi infection.

The term “favours treatment of the respiratory condition” means that the agent may induce a response of the appropriate T helper cells to combat the infection or the agent may create an environment which is equivalent to the appropriate T helper cell response for treating or eradicating that infection. This includes activation of the appropriate T helper cell to either generate more cytokines or to proliferate and increase the numbers of T helper cells generating the appropriate cytokines. Alternatively, the agent may create an environment which reduces the inappropriate response and highlights or promotes the appropriate response to treat the infection. For instance, antibodies to the cytokines may be introduced to reduce their effect, and emphasize or promote other effects.

In a further preferred aspect of the present invention, there is provided a method of treating a NTHi infection, said method comprising administering an effective amount of a Th-1 response inducing agent which induces an equivalent Th-1 response which favours treatment of the NTHi infection.

In another aspect of the present invention, there is provided a method of identifying a condition which is characterised by a differing T helper cell response, said method comprising;

collecting a biological sample from a patient having a mild form of the condition, and from a patient having an aggressive form of the condition;

exposing the sample to an antigen characteristic of the condition;

incubating the sample for a period sufficient to induce a cytokine response; and

determining the cytokines induced.

In another aspect of the present invention, there is provided a composition when used for the treatment of a respiratory condition wherein said condition results from an infection by a pathogen and wherein said condition is characterised by a differing T helper cell response.

### FIGURES

FIG. 1 shows results where cells were pre-incubated with (c) or without (a) anti-MHC II antibody and then cultured with NTHi. CD69 positive T helper cells were gated (R3 in a) and their IFN-γ (R11) and IL-5 (R8) staining is shown.

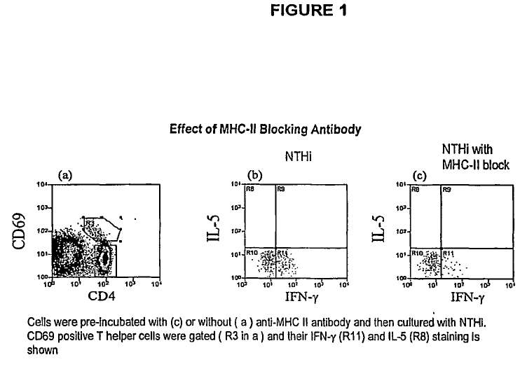

FIG. 2 shows a significant difference in IFN-γ and IL-4 production.

FIG. 3 shows responses to NTHi (a), Chest CT scan of one of the subjects with bronchiectasis and chronic NTHi infection with an acute exacerbation. It shows widespread destruction of lung tissue and associated pneumonia. (b), 9 samples of NTHi were obtained from sputum to make up a pooled antigen which was used to test immune response. The 9 different samples of NTHi were analyzed for their outer membrane proteins, which all appeared to be different. (c), Total immunoglobulin levels to NTHi were measured by ELISA in 13 control subjects and 13 bronchiectatic subjects. (d), The level of total immunoglobulin was the same in normal controls and subjects with bronchiectasis.

FIG. 4 shows T_{H} cytokine production in control & bronchiectasis subjects (a), IFN-γ production, (P<0.0001). (b), IL-4 production, (P<0.05). (c), IL-2 production, (P<0.005). (d), IL-10 production.

FIG. 5 shows histograms of T_{H} cell responses to NTHi in control & bronchiectasis subjects (a), shows gate R3 around cells staining for CD4⁺ and CD69⁺ in control subject and bronchiectasis subject. (b), shows staining of CD4⁺69⁺ cells for: 1) IL-4 and IL-2, 2) IL-10 and IFN-γ, 3) CD40L and IFN-γ.

FIG. 6 shows CD40L and CD40L/IFN-γ expression in control & bronchiectasis subjects (a), CD 40L expression, (P<0.0005). (b), CD 40L/IFN-γ expression, (P<0.0001).

FIG. 7 shows antibody responses to NTHi and cytokine responses to PPD in control & bronchiectasis subjects (a), IgG subclass end point titres to NTHi. Subjects with bronchiectasis (n=13) had significantly higher levels of IgG1 (P<0.05) and IgG3 (P<0.01) than controls (n=13). IgG4 levels were higher in the bronchiectasis group due to very levels of 3 subjects, but this did not achieve statistical significance. (b), Response to PPD, showing similar T_{H}1 responses in both groups.

### DESCRIPTION OF THE INVENTION

In a first aspect of the present invention there is provided a method of treating a respiratory condition wherein said condition results from an infection by a pathogen and wherein said infection is characterised by a differing T helper cell response, said method including administering an effective amount of an agent to induce an equivalent T helper cell response which favours treatment of the respiratory condition.

The term “treating” is used herein in its broadest sense and includes arresting or alleviating the infection to prevent further infection. For those patients which are susceptible to infections, or are likely to have chronic infections such as in a congenital case, the term “treating” also includes prevention.

The term “equivalent T-helper response” is a response which is similar or provides a state which results in a similar outcome to a T-helper cell response.

The condition to be treated is a respiratory condition, most preferably it is a respiratory condition of the upper or lower respiratory tract or both. This includes infections of the ear, nose and throat as well as the lungs.

Preferably the respiratory condition is chronic lung disease such as COPD, cystic fibrosis, bronchiectasis, tonsillitis, emphysema, ear infections or asthma.

All of these conditions in their chronic form can be identified by standard symptoms including shortness of breath, coughing and severe bronchitis.

Most preferably, the respiratory condition is bronchietasis. This is characterised by persistent and progressive dilation of bronchi or bronchioles as a consequence of the lung infection and inflammation, obstructive or congenital abnormalities. Symptoms generally characteristic to this condition include fetid breath, paroxymal coughing with the expectoration of mucopurulent matter. This condition may affect the bronchioles uniformly or occur in irregular pockets or the dilated bronchi may have terminal bulbous enlargements.

The infection which causes the respiratory condition will affect a T helper cell response. Most preferably the T helper cell response is a cytokine response. The T helper cell response should be a differing response where there exist at least two forms of T helper cell response. One form may be a Th-1 response and another form may be a Th-2 response. Importantly, with each respiratory condition there is preferred a dominant T helper cell response which can be changed to another dominant response. For instance, a dominant Th-1 response may be changed to a dominant Th-2 response.

A Th-1 response is an aquired immune response whose most prominent feature is a high cytotoxic T lymphocyte activity relative to the amount of of antibody production. A Th-1 response is defined as having more than 0.01% of the T helper cells producing IFN-γ, IL-2, IL-12 or IL-18 with no detectable IL-4 and IL5/10. The Th-1 response is promoted by CD4⁺ Th-1 T-helper cells. The Th-1 helper cells are a subset of helper-induced T lymphocytes which synthesize and secrete interleukin-2, gamma-interferon, interleukin-12 and interleukin-18.

The Th-2 response is an aquired immune response whose most prominent feature is high antibody production relative to the amount of cytotoxic T lymphocyte activity. A Th-2 response is defined as having more than 0.01% of the T helper cells producing IL-4 and IL-5/10 with no detectable IFN-γ, IL-2, IL-12 or IL-18. The Th-2 response is promoted by CD4+ Th-2 T-helper cells. The Th-2 cells are a subset of helper inducer T-lymphocytes which synthesize and secrete interleukins IL-4, IL-5, IL-6 and IL-10. These cytokines influence B-cell development and antibody production as well as augmenting humoral responses.

In the present invention, the method is applicable to a condition which is associated with an infection that is characterised by a differing T helper cell response. Preferably the differing T helper cell response is a difference between a Th-1 and a Th-2 response. These response are characterised by their cytokine profiles and patterns of immune effectors.

Generally, the Th-1 response is associated with a mild controlled disease whereas the Th-2 response is associated with a more aggressive form of the disease. Often, the aggressive form may be fatal.

The pathogen which causes the infection may be non-typable haemophilus influenzae (NTHi). NTHi is an important respiratory pathogen which in the context of infection is often found intracellularly. Applicants have found that most normal people have been infected by this bacterium and have made a clearing immune response with the production of T_{H}1 cytokines and CD40L. In contrast, patients with bronchiectasis and persistent infection with NTHi had an adaptive immune response characterized by T_{H}2 predominance, decreased CD40L production and higher levels of IgG1 and IgG3. These findings have relevance to both the pathogenesis and treatment of bronchiectasis, and perhaps to other conditions characterized by chronic respiratory infection with NTHi.

Most preferably, the pathogen is NTHi which causes any one of the conditions selected from the group including COPD, cystic fibrosis, bronchiectasis, emphysema/bronchitis, ear infection, asthma, sinusitis or tonsillitis. Even more preferred, the infection is NTHi causing bronchiectasis.

It is preferred that the respiratory condition is caused by an infection which has at least a mild and aggressive form characterised by a differing T-helper cell response for either form. Most preferably the response is a Th-1 or a Th-2 response for the mild or aggressive form respectively.

In a preferred aspect, there is provided a method of treating a NTHi infection, said method comprising administering an effective amount of an agent which induces an equivalent T helper cell response which favours treatment of the NTHi infection.

Without being restricted by theory, it is postulated by the applicants that for a NTHi infection, the normal response expected is a protective Th-1 response and that chronic infection may be associated with a non-clearing Th-2 response. However, applicants have found that for NTHi infection, the Th-2 response is predominant.

Preferably, the patient has a normal immune system which may be identified by screening for immune deficiency including considering lymphocyte subsets and proliferation, neutrophil phagocytosis and oxidative burst, immunoglobulins and complement. These are compared against a normal response where no detectable infection has occurred. It has been found that in a number of chronic bronchiectasis patients, the immune system is normal.

The agent used must be suitable for favouring a T helper-cell response which will be beneficial for treating the conditions.

The term “favours treatment of the respiratory condition” means that the agent may induce a response of the appropriate T helper cells to combat the infection or the agent may create an environment which is equivalent to the appropriate T helper cell response for treating or eradicating that infection. This includes activation of the appropriate T helper cell to either generate more cytokines or to proliferate and increase the numbers of T helper cells generating the appropriate cytokines. Alternatively, the agent may create an environment which reduces the inappropriate response and highlights or promotes an appropriate response to treat the infection. For instance, antibodies to the cytokines may be introduced to reduce their effect and to emphasize or promote other effects.

Where the response is a Th-2 response and a Th-1 response is desired, to favour treatment, it is preferred to administer an agent which causes an increase in cytokines typical of a Th-1 response or cause an increase in the number of Th-1 cells. Alternatively, antibodies or agents to remove cytokines typical of the Th-2 response may be used.

Preferably, where a Th-1 response is desired, the agent may be selected from the group including interferon, IL-2, IL-12, IL-18, blocking antibodies to IL-4, IL-5, or IL-10 or any agent which results in favouring a Th-1 response including antagonists of IL-4, IL-5, or IL-10.

More preferably, for favouring a Th-1 response, the agent is IFN-γ.

Accordingly, in a further preferred aspect of the present invention, there is provided a method of treating a NTHi infection, said method comprising administering an effective amount of a Th-1 response inducing agent which induces an equivalent Th-1 response which favours treatment of the NTHi infection.

A suitable Th-1 response inducing agent may be selected from the group including interferon, IL-2, IL-12, IL-18, blocking antibodies to IL-4, IL-5, or IL-10 or any agent which results in favouring a Th-1 response including antagonists of IL-4IL-5, or IL-10. Most preferably, the agent is IFN-γ.

The agent may be administered in any form which preferably reaches the site of infection. However, administration may be achieved intramuscularly, intravenously, intranasally, subcutaneously, intraperitoneally, intradermally, by infusion, suppository, implants or orally. The mode of administration is best selected for the type of infection. For instance in respiratory conditions, an intranasal form is most preferred. Aerosolized forms may be administered to optimise delivery such as including droplets of aerosol in a size range which allows for deposition in the respiratory tract.

For a respiratory condition such a bronchiectasis caused by a chronic infection of NTHi, a most suitable form of treatment includes inhaled interferon (IFN-γ). However, other Th-1 response inducing agents may be used.

The amounts and administration regimes suitable for treatment may be determined by the skilled addressee based on the severity of the infection. Suitable amounts will depend on the mode of administration. Intranasal or aerosol IFN may be used in the order of 250 to 1000 μg per dose. This may be administered daily or for at least 3 days to treat a respiratory infection, depending on the severity of the infection. In an aerosol a suitable amount may be approximately 20 μg/litre of air. The appropriate amounts may be deduced by the skilled clinician and my also be measured by macrophage responses such as mRNA or oxidative burst.

The agent may be administered alone or in combination with other forms of treatment of the respiratory condition. Such other forms include the administration of antibiotics such as amoxycillin. However, the appropriate antibiotics will depend on the infection to be treated.

Preferably, to treat NHTi infection it is desired to use IFN-γ in conjunction with amoxicillin.

Intracellular pathogens associated with a spectrum of clinical disease and immune responses include Leishmania major and Mycobacterium leprae. In both these infections host protective responses have been shown to be T_{H}1 predominant, while T_{H}2 responses with decreased CD40L production (lepromatous leprosy and visceral leishmaniasis) are associated with progressive infection. As well, patients with lepromatous leprosy and visceral leishmaniasis produce higher levels of antigen specific IgG1 and IgG3 than controls. In both these infections controlling immune responses are Th1 predominant (i.e. production of IFN-γ, CD40L etc), while Th2 responses are associated with progressive disease. The non-clearing Th2 responses in both these conditions are associated with high levels of the antibody subclasses IgG1 and IgG3. Similarly in the subjects with bronchiectasis they had high levels of IgG1 and IgG3.

In leprosy and leishmaniasis where subjects do not show a clearing immune response, cytokine therapy can be helpful. Trials have shown that cytokines, particularly in combination with other agents such as antibiotics may cause clearing of the infectious agent. Such a combination could be effective in patients with bronchiectasis, who have intractable symptoms despite full medical therapy. Inhaled IFN-γ, which in normal subjects is easy to administer and produces potent activation of pulmonary macrophages with no systemic side effects, is a potential option. The use of Th1 cytokines such as IFN-γ or IL18 in conjunction with antibiotics for the treatment of infections with nontypable Haemophilus influenzae is preferable. The most efficious way may be to use inhaled IFN-γ.

In another aspect of the present invention, there is provided a method of diagnosing a respiratory condition which is characterised by a differing T helper cell response, said method comprising;

collecting a biological sample from a patient having a mild form of the condition,

collecting a biological sample from a patient having an aggressive form of the condition;

exposing the samples to an antigen characteristic of the condition;

incubating the samples for a period sufficient to induce a cytokine response; and

determining and comparing the cytokines induced.

Preferably, the method diagnoses an aggressive form of the respiratory condition characterised by a differing T helper cell response. Preferably, the difference is between a Th-1 and a Th-2 response.

The biological sample may be any sample from a patient which may include T helper cells. Preferably, the sample will include both Th-1 and Th-2 cells. Most preferably, the biological sample is whole blood. The sample may be isolated lymphocytes. The lymphocytes may be isolated by any method available such as on a ficoll gradient or by centrifugation to obtain a buffy coat enriched with lymphocytes.

The sample is exposed to an antigen which is characteristic of the condition. For most infections, exposing the sample to an organism which causes the infection will be sufficient for this step. For instance, where the infection is caused by a NTHi infection, exposing the sample to whole NTHi will be sufficient. If a characteristic antigen is identifiable to the infection causing agent, then this may be used.

The period sufficient to activate a cytokine response may vary depending on the antigen used. A period of approximately 4 to 8, preferably 6 hours would be sufficient. However, the period may be arbitrary providing the biological samples from both the mild and aggressive forms are treated concurrently.

The cytokines to be determined will be those that reflect a T-helper cell response. Preferably, the cytokines determined include IFN-γ, IL-2, IL-12, IL-18, IL-4, IL-5, and IL-10. The cytokines IFN-γ, IL-2, IL-12 or IL-18 are characteristic of a Th-1 response and the IL-4, IL-5 and IL-10 are characteristic of a Th-2 response. Preferably, the IFN-γ is determined and compared.

In a preferred aspect of the invention, the method further includes:

incubating the biological sample with an agent to prevent exportation of cytokines from the biological sample prior to determination of induced cytokines.

The method seeks to determine intracellular cytokines which are induced in the presence of the antigen. By using a exportation blocking agent, the cytokines may be contained prior to their determination. Preferably, a Golgi blocking agent is used. More preferably, Brefeldin A is used.

In a further preferred aspect of the invention, the method further includes:

exposing the biological sample to a permeablizing agent to release the induced cytokines prior to determination.

An agent which causes the cells to become permeable may be used. A suitable agent is saponin.

The cytokines may be determined by any method that identifies and distinguishes the cytokines. Labeled antibodies may be used especially immunofluorescent antibodies. These may be added at the time of permeablizing the cells in the biological sample.

Detecting the labeled cytokines may be conducted in any manner. However, flow cyometry is particularly preferred.

In another aspect of the present invention, there is provided a composition when used for the treatment of a respiratory condition wherein said condition results from an infection by a pathogen and wherein said condition is characterised by a differing T helper cell response said composition including an agent which can induce an equivalent T helper cell response which favours treatment of the respiratory condition.

Preferably, the agent is interferon, most preferably it is IFN-γ or a component which behaves in a similar manner to IFN. It may be an IFN-like compound. The agent, preferably IFN-γ may be naturally produced or provided in recombinant form providing the compound behaves in the same manner as IFN-γ to induce a T helper cell response which preferably can displace emphasis from a Th-2 response to a Th-1 response.

The composition may include at least one pharmaceutically acceptable carrier/or diluent along with the agent. The carrier is selected for exhibiting excellent prophylactic or therapeutic activity. Ideally, the carrier or diluent is selected for convenient administration as outlined above. Such carriers will be familiar to those skilled in the art.

Suitable amounts will depend on the amelioration of the infection and will depend on the particular condition to be treated.

In a preferred aspect, the present invention provides a composition when used for the treatment of a NTHi infection, said composition including an effective amount of IFN-γ or an IFN-γ-like compound and a carrier.

The “effective amount” is an amount which is useful for the treatment of the infection.

It is preferred that the composition include sufficient IFN-γ to deliver an effective amount to treat the infection. Suitable amounts may include 250 to 1000 μg of IFN-γ per dose. The composition may also include in an aerosol composition an amount of 20 μg/litre air.

The present invention will now be more fully described with reference to the following examples. It should be understood, however, that the description following is illustrative only and should not be taken in any way as a restriction on the generality of the invention described above.

### EXAMPLES

### Example 1

### Identification of T Helper Cell Response in Bronchiectasis

The measurement of Th cell cytokine responses to antigens has generally been done by isolation and cloning. This technique has the significant disadvantage of being extremely difficult to perform and often takes years (if successful) to get results.

An antigen specific, flow cytometric technique to measure intracellular cytokine production was used. The method is performed by adding antigen (e.g. CMV) and co-stimulatory antibodies to whole blood, which is incubated for 6 hours. A Golgi blocking agent (Brefeldin A) is added, to prevent the cytokines from being exported outside the Th cell. The cells are then permeablised with saponin and immunofluorescent antibodies are added for: 1) Activated Th cells-CD4, CD69, 2) Cytokines-IFN-γ, IL2, IL4, IL5/IL10. Four colour analysis is then performed by flow cytometry.

1) The response to CMV antigen was considered in 10 controls and was compared to the response to NTHi antigen in 6 controls. The CMV was obtained from Biowhitaker (USA) and the heat-inactivated, sonicated NTHi was obtained from a child with conjunctivitis.

2) The response to NTHi in 13 subjects with radiologically confirmed bronchiectasis, aged 55±11 yrs (9 female, 4 male) with 21 controls was compared. All the bronchiectatic subjects had been screened for immune deficiency (lymphocyte subsets & proliferation; neutrophil phagocytosis & oxidative burst, immunoglobulins; complement) and cystic fibrosis; and only subjects with normal results were selected for this study. The bronchiectatic subjects had all had NTHi isolated from their sputum previously on multiple occasions (average 3 times over past 4 years). The NTHi antigen used was pooled from 10 isolates from sputum samples grown on agar plates. So that a relevant antigen would be used; 3 of the NTHi were obtained from bronchiectatics, 4 from COPD patients and 3 from subjects with normal lungs. The NTHi obtained was heat inactivated, sonicated and suspended at a concentration of 2.0 McFarlane. The number of specific activated cells expressing CD69 and CD40 ligand was also measured. The responses of both groups to PPD (purified protein derivative of Tuberculosis) was also measured.

Subjects for each cytokine; had 150, 000 (average) individual Th cells analysed. Th 1 responders were defined as having more than 0.010% of their Th cells producing IFN-γ with no detectable IL4 and IL5/10. Th2 responders were defined as having more than 0.010% of their Th cells producing IL4 and IL5/10 with no detectable IFN-γ.

TABLE 1
Th cytokine response to CMV and NTHi in control subjects

| | | Th1 responders | Th2 (IL4, IL5) |
|---|---|---|---|
| Subject group | No of subjects | (IFNγ) | responders |
| CMV controls | 10 | 5 | 0 |
| NTHi controls | 6 | 3 | 0 |

There was a distinct Th1 response in both the CMV and the NTHi subject groups, with Th cells producing IFN-γ but undetectable levels of IL4 and IL5. To further validate this response, the effect of a blocking antibody HLA-DR (DP and DQ were not available) of the major histocompatibility complex (MHC-II) was tested. This resulted in a 74% reduction in the number of Th cells producing IFN-γ, confirming the role of the Th cell.

TABLE 2
Th cytokine response to NTHi in Controls and
Bronchiectatics

| | No | Th1 responders | Th2 (IL4, IL10) |
|---|---|---|---|
| Subject group | of subjects | (IFNγ) | responders |
| NTHi controls | 21 | 10 | 0 |
| NTHi bronchiectatics | 13 | 0 | 7 |

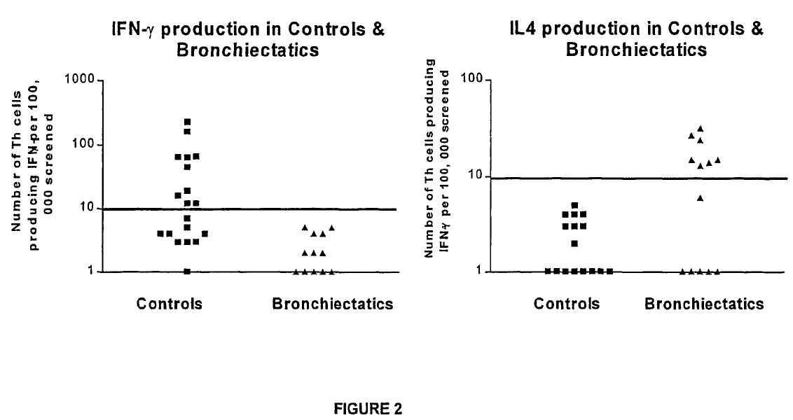

There was a distinct Th1 response by the control group to NTHi. This was quite different to the Th2 response produced by the bronchiectatics. These results are shown in FIG. 2. ( a log scale is used

There was a statistically significant difference between controls and bronchiectatics; using rank sum, p<0.0001.

To validate these findings; they were reproducible on re-testing and could be prevented by MHC-II blocking antibodies. In addition, the expression of CD40 ligand in association with the Th1 responses could be measured.

The response of a subgroup of controls and bronchiectatics for their response to PPD was also tested. 7/8 controls and 5/5 bronchiectatics made a Th1 response to PPD.

The results show that bronchiectasis appears to be associated with an isolated Th2 response to NTHi, compared with the Th1 response from normal controls. Possible explanations for this difference are:

i) A genetic predisposition to produce Th2 responses to NTHi.

ii) Chronic infection with NTHi may be associated with a change in the immune response.

Differing Th1/Th2 responses have been described in several infectious diseases (Leishmaniasis, Schistosomiasis & Leprosy). In leprosy a Th1 response is associated with mild controlled disease against this intracellular organism, while a Th2 response (where antibody is produced that is ineffective against this intracellular organism) is associated with aggressive fatal disease. Cytokine therapy with IFN-γ can cause a switch from a Th2 to a Th1 response with substantial clinical improvement. Inhaled IFN-γ, with its direct effect on pulmonary macrophages, with minimal side effects, may have much to offer, in patients with chronic upper & lower respiratory tract infection with NTHi.

Accordingly, it is concluded that bronchiectasis is associated with a non-clearing Th2 response to NTHi, which is distinctly different from the Th1 response produced by normal controls. These findings may have significant relevance in regard to both pathogenesis and potential treatments for bronchiectasis. These findings may also have relevance to other conditions characterised by chronic NTHi infection, such as COPD and tonsillitis.

### Example 2

### Non-Clearing Immune Responses to Nontypable Haemophilus influenzae are Associated with Chronic Infection in Bronchiectasis

The adaptive immune response to NTHi in healthy controls and patients with bronchiectasis was tested. Subjects in both groups had a strong antibody response to this bacterium. Using flow cytometry to measure antigen specific intracellular cytokine production, it was established that hormal controls made a T_{H}1 response to NTHi, while subjects with bronchiectasis made a T_{H}2 response and had significantly less production of CD40 ligand (CD40L). The bronchiectasis group also had higher levels of antigen specific IgG1 and IgG3, and was atopic.

(a) Subjects with Idiopathic Bronchiectasis had Recurrent Isolation of NTHi from their Sputum

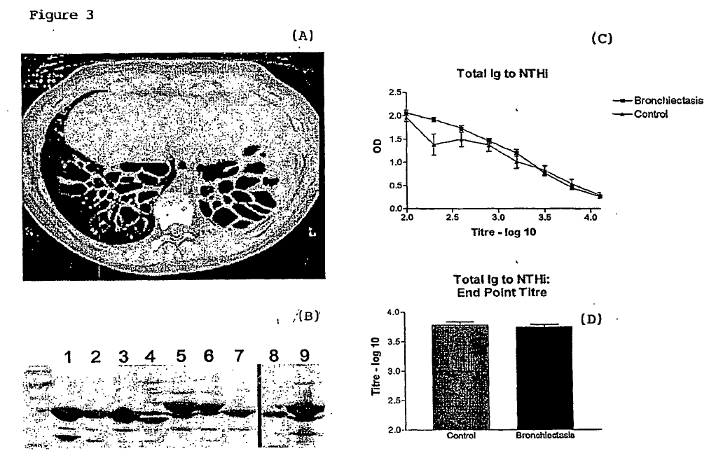

A cohort of 15 subjects with bronchiectasis, who had recurrent isolation of H. influenzae from their sputum, was studied. Subjects had severe symptoms with daily sputum production and fatigue, and significant destruction of lung tissue (FIG. 3a). All had a comprehensive clinical assessment, screen of their immune function (Table 3) and mutation analysis for cystic fibrosis; and were classified as having idiopathic bronchiectasis. The subjects had all had multiple significant isolates of NTHi from their sputum in the past 5 years, with an average of 4 significant isolates (defined as the presence of plentiful gram negative cocco-bacilli, polymorphs and a moderate to profuse growth of NTHi). In all subjects NTHi was the main bacteria isolated from their sputum and in most cases the only bacteria.

TABLE 3

| | | |
|---|---|---|
| | Subjects with | |
| | bronchiectasis | Control values |
| Units | (mean & SD) | (mean & SD) |

Immune function of subjects with bronchiectasis Subjects
were immuno-competent. The level of IgG2 was a little lower
than controls but all subjects were in the normal range. Two
of the subjects had low CD4⁺ counts (30% and 37%),
and 1 subject had a low lymphocyte proliferation (720).

| | | | |
|---|---|---|---|
| White cell count & | | | |
| differential | | | |
| White cells | ×10˜9/L | 7.1 ± 2.0 | 7.5 ± 1.8 |
| Neutrophils | ×10˜9/L | 4.7 ± 1.8 | 5.0 ± 1.5 |
| Lymphocytes | ×10˜9/L | 1.7 ± 0.4 | 2.1 ± 0.7 |
| Monocytes | ×10˜9/L | 0.5 ± 0.2 | 0.6 ± 0.1 |
| Eosinophils | ×10˜9/L | 0.2 ± 0.1 | 0.2 ± 0.1 |
| Immunoglobulins | | | |
| IgG | g/L | 11.6 ± 3.2 | 11.5 ± 4.5 |
| IgA | g/L | 2.8 ± 1.1 | 2.4 ± 0.8 |
| IgM | g/L | 1.4 ± 0.8 | 1.4 ± 0.5 |
| IgG1 | g/L | 8.1 ± 2.2 | 8.6 ± 2.2 |
| IgG2 | g/L | 3.0 ± 1.1 | 4.1 ± 1.2 |
| IgG3 | g/L | 1.0 ± 0.4 | 0.8 ± 0.4 |
| IgG4 | g/L | 0.4 ± 0.3 | 0.4 ± 0.3 |
| Complement | | | |
| C3 | g/L | 1.27 ± 0.27 | 1.35 ± 0.23 |
| C4 | g/L | 0.31 ± 0.10 | 0.30 − 0.08 |
| Lymphocyte Subsets- | | | |
| T_{H} (CD3+/CD4+) | % | 44 ± 12 | 45 ± 6 |
| T_{c} (CD3+/CD8+) | % | 29 ± 8 | 25 ± 8 |
| B cell (CD19+) | % | 10 ± 4 | 13 ± 5 |
| NK cells (CD3−/CD16+) | % | 11 ± 3 | 12 ± 6 |
| NK cells (CD3−/CD56+) | % | 10 ± 3 | 11 ± 5 |

Lymphocyte Proliferation/³H thymidine uptake

| | | |
|---|---|---|
| Normal range: >1000 | Subjects below | 1 (value |
| CPM/1000 Lymph. | normal range: | of 720) |

(b) Controls and Subjects with Bronchiectasis all had Detectable Antibody to NTHi

NTHi is a heterogeneous species, with multiple outer-membrane proteins sub-types. The sero-conversion rate to NTHi of the general population is not known. To assess the antibody response a pooled NTHi antigen was prepared (FIG. 3b) from multiple isolates and subtypes. Using ELISA the total immunoglobulin (Ig) to NTHi antigen was measured in; subjects with bronchiectasis and chronic infection with NTHi (n=13), and controls (n=13). It was found that both controls and subjects all had significant titres of immunoglobulin to NTHi, which were of similar end point (FIG. 3c). Normal healthy people and the subjects' with bronchiectasis have the same titre of antibody to the nonypapble Haemophilus influenzae (NTHi). This suggests that most normal people have had recent infection with this bacteria which has been cleared by their immune response.

(c) Measurement of Antigen Specific T Helper Cell Responses

The measurement of antigen specific T_{H} responses is difficult due to the low frequency of these cells. Even in the case of repeated CD4⁺ antigenic stimulation with allergen in an atopic individual, clone frequency has been estimated to remain as low as 1 in 10⁵. Until recently assessment of the predominance of T_{H}1/T_{H}2 responses has usually been performed by isolation and cloning of the antigen specific T_{H} cells. However new techniques have become available such as the Elispot assay, MHC tetramers and the measurement of antigen specific intracellular cytokines by flow cytometry. The flow cytometry method has the advantage that multiple different parameters can be measured in cells simultaneously; and has been used to confirm a T_{H}1 response to Cytomegalovirus by analyzing peripheral blood samples.

The flow cytometry technique was adapted to establish the nature of the T_{H} cell response and CD40L production to NTHi in normal control subjects, and subjects with bronchiectasis and chronic infection with NTHi. For each cytokine an average of 100 000 CD4⁺ cells (with a minimum of 50 000 CD4⁺ cells) were screened. Cytokine production by activated T_{H} cells (CD4⁺ CD69⁺) was distinct and reproducible when at least 10 per 100 000 CD4⁺ lymphocytes (i.e. ≧0.010%) produced one cytokine.

T_{H} responses were defined as:

1) T_{H}1 response: ≧10 per 100 000 CD4⁺ cells screened staining for CD69 and IFN-γ, and <10 per 100 000 CD4⁺ cells screened staining for CD69 and IL-4.

2) T_{H}2 response: ≧10 per 100 000 CD4⁺ cells screened staining for CD69 and IL-4, and <10 per 100 000 CD4⁺ cells screened staining for CD69 and IFN-γ. Other responses were classed as indeterminate.

(i) Normal control subjects made a T_{H}1 predominant response to NTHi

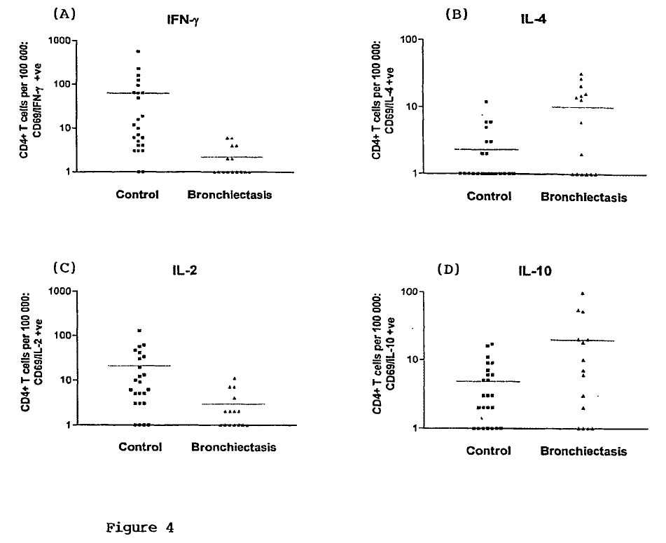

The antigen specific responses of the normal controls were characterized by the predominance of the T_{H}1 cytokine IFN-γ; in whom levels were significantly higher (P<0.0001) than bronchiectatic subjects (FIG. 4a). Of the 24 control subjects, half (12) of them made a Th1 response. No control subject made a T_{H}2 response. Testing for other cytokines IL-2 and IL-10, mirrored these responses (FIGS. 4c and 4d). In 8 out of the 12 subjects classified as having a T_{H}1 response there was comparable production of IL-2 (i.e. >0.010% of CD4⁺ cells staining for CD69 and IL-2), and lack of production of IL-10.

Antigen is presented to its specific T helper cell by an antigen presenting cell (APC) in association with MHC-II. The addition of MHC-II blocking antibody (to HLA-DR) prevented the expression of cytokine production by the T_{H} cells consistent with antigen specific responses.

(ii) Subjects with bronchiectasis and persistent infection with NTHi did not make a T_{H}1 response and instead made a T_{H}2 response

The antigen specific responses of the bronchiectatic subjects were characterized by the predominance of the T_{H}2 cytokine IL-4, in whom levels were significantly (P<0.05) higher than normal controls (FIG. 4b).

Using the described criteria for T_{H} cell response, of the 15 subjects with bronchiectasis and chronic infection with NTHi, none made a T_{H}1 response and 7 made a T_{H}2 response with IL-4 production. Testing for other cytokines i.e. IL-2 and IL-10 mirrored these responses, with a lack of IL-2 production and 6 out of 7 T_{H}2 responders having clear production of IL-10. One of the other bronchiectatic subjects did not have detectable staining for IFN-γ, IL-2 or IL-4, but had significant production (0.098%) of IL-10 by CD4⁺ CD69⁺ cells, consistent with a T_{H}2 response. Thus over half of the subjects (8 out of 15) with bronchiectasis made a T_{H}2 response to NTHi.

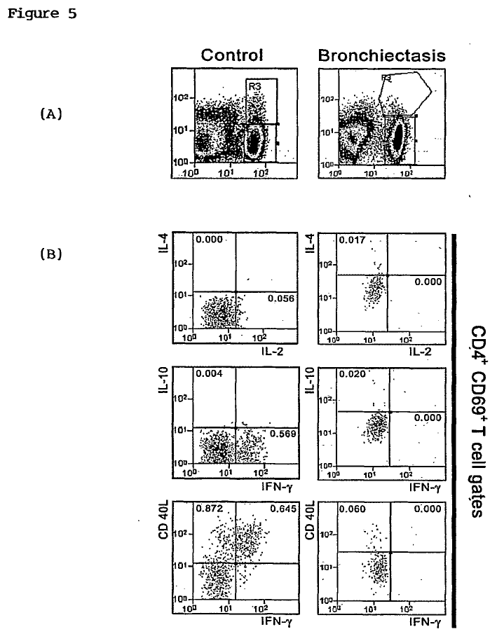

Examples of T_{H}1 and T_{H}2 responses are shown in FIG. 5.

The bronchiectatic subjects who had chronic non-clearing infection with NTHi, made a completely different immune response. None of these patients made a T_{H}1 response. Instead over half the group with bronchiectasis made a T_{H}2 response. Such a response would not be protective against an invasive intracellular pathogen.

(iii) The subjects with bronchiectasis had decreased production of CD40 ligand

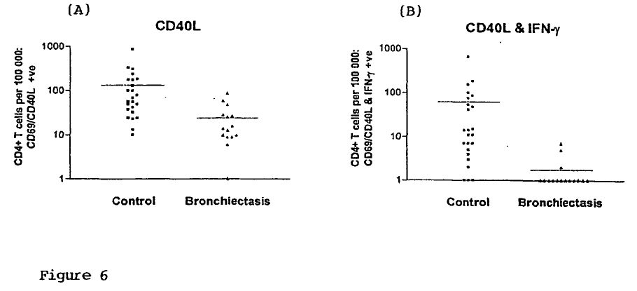

CD40L is expressed by activated CD4⁺ cells to signal to the APC and with IFN-γ is crucial in mediating T_{H}1 responses. The production of CD40L was significantly (P<0.001) higher in normal controls than the subjects with bronchiectasis (FIG. 6a). Normal controls also produced high levels of IFN-γ in association with CD40L (P<0.0001) than bronchiectatic subjects (FIG. 6b).

The expression of CD40L, particularly in association with IFN-γ was significantly lower in the bronchiectatic group and this would be associated with decreased activation of macrophages.

The expression of CD40 ligand (CD40L) is more associated with Th1 than Th2 responses.

(d) Subjects with Bronchiectasis were Atopic and Produced High Titres of IgG1 and IgG3 to NTHi

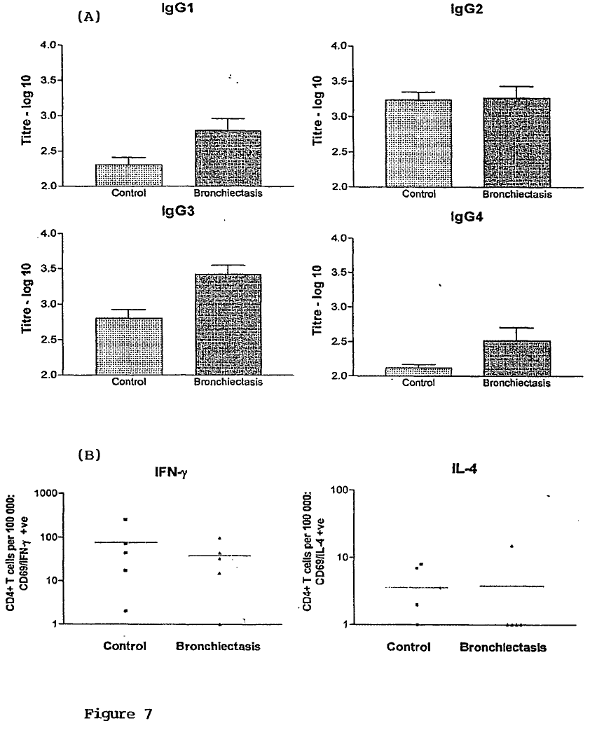

The group of bronchiectatic subjects had a high incidence of asthma and allergy, and 7 had moderately elevated IgE levels (range: 103-378 IU/ml; normal value<100 IU/ml). The IgG subclass production to NTHi was measured by ELISA and the levels of IgG1, (P<0.05); and IgG3, (P<0.01); made by the subjects with bronchiectasis were significantly elevated compared with controls (FIG. 7a).

This suggests these subjects as a group are allergic and therefore more prone to make Th2 Responses.

(e) Controls and Subjects with Bronchiectasis made Similar T_{H}1 Responses to Tuberculosis Antigen

The hypothesis that patients with bronchiectasis had a generalized defect in their antigen specific responses was tested. The response to purified protein derivative of tuberculosis (PPD) antigen, is expected to be a T_{H}1 response. The response to PPD in controls and subjects with bronchiectasis who—had previously received BCG vaccination was assesed.

Blood was taken from 5 controls and 5 subjects with bronchiectasis and incubated with PPD. Antigen specific responses were measured by flow cytometry. Both the controls and the bronchiectatic subjects produced a similar T_{H}1 response (FIG. 7b).

Finally it is to be understood that various other modifications and/or alterations may be made without departing from the spirit of the present invention as outlined herein.

### References

1. Suni M A et al. Detection of antigen-specific T cell cytokine expression in whole blood by flow cytometry. J Immunol Methods 1998 March 1; 212:89-98

2. Yamamura M et al. Defining protective responses to pathogens: cytokine profiles in leprosy lesions. Science 1991, 254:277-279

3. Janeway C A. Manipulating the immune response to infection p573. In: Immunobiology, the immune system in health and disease. Eds; Janeway, Travers, Walport, Capra. Garland, New York, 1999

1. A method of treating a respiratory condition wherein said condition results from an infection by a pathogen and wherein said infection is characterised by a differing T helper cell response, said method including administering an effective amount of an agent to induce an equivalent T helper cell response which favours treatment of the respiratory condition.

2. A method according to claim 1 wherein the respiratory condition is a condition of the upper or lower respiratory tract or both.

3. A method according to claim 1 or 2 wherein the respiratory condition is selected from the group including chronic lung disease, COPD, cystic fibrosis, bronchiectasis, tonsillitis, emphysema, ear infections or asthma.

4. A method according to anyone of claims 1 to 3 wherein the respiratory condition is caused by an NTHi infection.

5. A method according to claim 4 wherein the condition is bronchietasis.

6. A method according to anyone of claims 1 to 5 wherein the T-cell helper response is a differing cytokine response.

7. A method according to anyone of claims 1 to 6 wherein the T-cell helper response is a difference between a Th-1 and a Th-2 response.

8. A method according to anyone of claims 1 to 7 wherein the agent induces a Th-1 or Th-2 response.

9. A method according to claim 8 wherein the agent to induce a Th-1 response is selected from the group including IFN-γ, IL-2, IL-1, IL-18 or an agent which reduces the effect of IL-4, IL-5, IL-6 or IL-10 including antagonists of IL-4, IL-5, IL-6 or IL-10.

10. A method according to claim 8 wherein the agent to induce a Th-2 response is selected from the group including IL-4, IL-5, IL-6 or IL-10 or an agent which reduces the effect of IFN-γ, IL-2, IL-1, IL-18 including antagonists of IFN-γ, IL-2, IL-1, IL-18.

11. A method according to anyone of claims 4 to 9 wherein the agent is IFN-γ.

12. A method according to claim 11 wherein the IFN-γ is administered at 250 to 1000 μg per dose.

13. A method according to claim 12 wherein the IFN-γ is an aerosol at approximately 20 μg/litre of air.

14. A method according to anyone of claims 1 to 13 wherein the agent is administered with an antibiotic.

15. A method according to claim 14 wherein the antibiotic is amoxycillin.

16. A method of diagnosing a respiratory condition which is characterised by a differing T helper cell response, said method comprising;
collecting a biological sample from a patient having a mild form of the condition,
collecting a biological sample from a patient having an aggressive form of the condition;
exposing the samples to an antigen characteristic of the condition;
incubating the samples for a period sufficient to induce a cytokine response; and
determining and comparing the cytokines induced.

17. A method according to claim 16 which diagnoses an aggressive form of the respiratory condition characterised by a different Th-1 and Th-2 response between the aggressive and the mild form of the condition.

18. A method according to claim 16 or 17 wherein the cytokine response is measured by the presence of a cytokine selected from the group including IFN-γ, IL-2, IL-1, IL-18, IL-4, IL-5, IL-6 or IL-10.

19. A method according to anyone of claims 16 to 18 further including incubating the biological sample with an agent to prevent exportation of cytokines from the biological sample prior to determination of induced cytokines.

20. A method according to claim 19 wherein the agent to prevent exportation of cytokines from the biological sample is Brefeldin A.

21. A method according to anyone of claims 16 to 20 further including exposing the biological sample to a permeablizing agent to release the induced cytokines prior to determination.

22. A method according to claim 21 wherein the permeablizing agent is saponin.

23. A composition when used for the treatment of a respiratory condition wherein said condition results from an infection by a pathogen and wherein said condition is characterised by a differing T helper cell response said composition including an agent which can induce an equivalent T helper cell response which favours treatment of the respiratory condition.

24. A composition according to claim 23 wherein the condition is caused by an NHTi infection.

25. A composition according to claim 23 wherein the condition is bronchiectasis.

26. A composition according to anyone of claims 23 to 25 wherein the agent is IFN-γ.

27. A composition according to claim 26 wherein the IFN-γ is 250 to 1000 μg per dose.
